(19) United States
(12) Patent Application Publication
(10) Pub. No.: US 2014/0073979 A1
(43) Pub. Date: Mar. 13, 2014

(54) eCard ECG Monitor

(71) Applicants: Salvatore Richard Inciardi, Manalapan, NJ (US); Sandra D. Elliott, Barnegat, NJ (US); Fredrik Einberg, Huddinge (SE); Kristian Svensson, Arsta (SE)

(72) Inventors: Salvatore Richard Inciardi, Manalapan, NJ (US); Sandra D. Elliott, Barnegat, NJ (US); Fredrik Einberg, Huddinge (SE); Kristian Svensson, Arsta (SE)

(21) Appl. No.: 13/860,323

(22) Filed: Apr. 10, 2013

Related U.S. Application Data
(60) Provisional application No. 61/622,566, filed on Apr. 11, 2012.

(51) Int. Cl. A61B 5/0404

(52) U.S. Cl. CPC A61B 5/0404
USPC 600/509

(57) ABSTRACT

Portable ECG (electrocardiograph) monitoring device combinations that records, transmits and displays sampled ECG data from handheld devices having sensors and integrated electronics housed in a card-like member for determining and displaying a user's processed ECG for medical diagnostic and informational purposes. The monitoring device of the present invention records and wirelessly transmits raw sampled ECG data from the handheld sampling device and optionally additional wireless ECG sensors to a remotely associated display device for processing and analyzing the raw data thereby shifting processing overhead from the handheld device to the display device.

### CROSS-REFERENCE TO RELATED PATENT APPLICATIONS

The present patent application claims the benefit of United Stated Provisional Patent Application No. 61/622,566, filed on Apr. 11, 2012, titled “E CARD ECG MONITOR”, the entirety of which is hereby incorporated by reference.

### FIELD OF THE INVENTION

The present invention relates to heart rate monitors and, in particular, to portable, handheld ECG (electrocardiograph) monitoring device combinations that record, transmits and display sampled ECG data that determines and displays the user's processed ECG for medical diagnostic and informational purposes. More specifically, the monitoring device of the present invention records and transmits raw sampled data to a remotely associated display device for processing the raw data thereby shifting processing overhead from the handheld device to the display device.

### BACKGROUND OF THE INVENTION

People exercise regularly in order to counteract the detrimental effect caused by the modern sedentary lifestyle. Often heart rate monitoring devices are used in fitness related activities for a variety of purposes including but not limited to fitness and conditioning, weight loss, goal-oriented heart rate training as well as for general cardiac monitoring. Heart rate is an important parameter which is indicative of the body conditions of a human being whether at rest or when exercising and other strenuous physical activity. Additionally, heart rate monitors may sometimes be employed by healthcare professionals for chronic and/or acute heart condition monitoring and/or diagnosis.

Ambulatory ECG monitoring provides a health care provider with a recording of a patient's heart electrical activity over a prolonged period. Basically, there are two variants of this type of monitoring: Holter monitoring and event monitoring. A Holter monitor is a portable device for continuously monitoring various electrical activity of the cardiovascular system for at least 24 hours (often for up to two weeks at a time). For patients having more transient symptoms, a cardiac event monitor which can be worn for a month or more can be used. With event monitoring, the device is worn for up to 7 days, but only records when the patient instruct it to do so.

Both monitors can record heart rate and rhythm when a patient feels chest pain or symptoms of an irregular heartbeat (called arrhythmia or sometimes dysrhythmia). A cardiologist can look at the recording around the time of the reported or observed event symptoms. This information can help clarify the presence and nature of any heart problem.

Heart rate monitoring devices are often expensive, and in some cases are cost prohibitive for many consumers. Although there have been a number of attempts at making such devices less expensive doing so has often been at the expense of accuracy and reliability. Additionally, in an attempt to make some of these devices more portable, these devices are nonetheless cumbersome to use, require multiple electrodes and associated wiring and are often too complicated and ill-suited for typical consumer use.

For example, during exercise, sports or athletic activities, it is often desirable to monitor the heart-rate for optimal results as well as for personal safety. The simplest way to measure heart-rate is probably by finger pressing the wrist and then counting the number of heart beats within a given time in order to calculate the heart beat per minute. However, this is a relatively primitive method which may not give an accurate result, often requires a relatively long pulse-counting period and may not be sufficiently reliable for most practical purposes. To facilitate more accurate and convenient heart-rate measurements, devices with electrocardiographic (ECG) signal processing and measuring means are available.

Muscle contraction in the human body is caused by electrical biosignals. Heart muscle contractions are caused by a biosignal referred to as the electrocardiogram (ECG) signal. ECG signals are electrical signals flowing through the heart which are indicative of the electrical activity of the heart. An ECG device detects and amplifies the tiny electrical changes on the skin that are caused when the heart muscle depolarizes during each heartbeat. At rest, each heart muscle cell has a negative charge (membrane potential) across its outer wall (or cell membrane). Increasing this negative charge towards zero (via the influx of the positive ions, Na+ and Ca++) is called depolarization, which activates the mechanisms in the cell that cause it to contract. During each heartbeat a healthy heart will have an orderly progression of a wave of depolarization that is triggered by the cells in the sinoatrial node, spreads out through the atrium, passes through “intrinsic conduction pathways” and then spreads all over the ventricles. This is detected as tiny rises and falls in the voltage between two electrodes placed either side of the heart which is displayed as a wavy line either on a screen or on paper. This display indicates the overall rhythm of the heart and weaknesses in different parts of the heart muscle.

Usually more than 2 electrodes are used and they can be combined into a number of pairs (For example: Left arm (LA), right arm (RA) and left leg (LL) electrodes form the three pairs LA+RA, LA+LL, and RA+LL). The output from each pair is known as a lead. Each lead is said to look at the heart from a different angle. Different types of EKGs can be referred to by the number of leads that are recorded, for example 3-lead, 5-lead or 12-lead ECGs (sometimes simply “a 12-lead”). A 12-lead ECG is one in which 12 different electrical signals are recorded at approximately the same time and will often be used as a one-off recording of an ECG, traditionally printed out as a paper copy. 3- and 5-lead ECGs tend to be monitored continuously and viewed only on the screen of an appropriate monitoring device, for example, during an operation or while being transported in an ambulance. There may or may not be any permanent record of a 3- or 5-lead ECG, depending on the equipment used.

This is one of the best ways to measure and diagnose abnormal rhythms of the heart, particularly abnormal rhythms caused by damage to the conductive tissue that carries electrical signals, or abnormal rhythms caused by electrolyte imbalances. For example, in a myocardial infarction (MI), the ECG can identify if the heart muscle has been damaged in specific areas, though not all areas of the heart are covered. The ECG cannot reliably measure the pumping ability of the heart, for which ultrasound-based (echocardiography) or nuclear medicine tests are used. It is possible for a human or other animal to be in cardiac arrest but still have a normal ECG signal (a condition known as pulseless electrical activity).

Each typical and complete ECG signal or electrocardiogram includes a complete waveform with the more salient labels P, Q, R, S and T indicating the more distinctive and significant features of the waveform. The QRS complex describes a region of particular activity in the ECG signal during each heartbeat. It is generally recognized that the P wave arises from the depolarization of the atrium, the QRS complex arises from depolarization of the ventricles, and the T-wave arises from re-polarization of the ventricle muscle. The magnitude of the tall, spiky R-wave of the PRS complex is approximately 1 mV. When the heart beats, a train of repetitive ECG signals with the characteristic P-QRS-T waveform can be detected. The instantaneous heart-rate can be determined from the period of the train of ECG signals, for example, by measuring the time difference between immediately adjacent spiky R-peaks of the train of the ECG signals.

In order that the heart-rate can be determined by automated ECG analysis for enhanced accuracy, sensitivity, convenience as well as within a shorter time, devices with automated ECG analysis capability are required. ECG signals are usually detected by applying electrodes to the skin, usually also in the presence of noise. Typical examples of noise sources which are commonly known to corrupt ECG signals include, for example, power line interference, electrode contact noise motion artifacts, muscle contraction (electrode myographic, EMG), based line drift and ECG amplitude modulation with respiration, instrumentation noise generated by electronic devices, electrosurgical noise and other, less significant noise sources. The nature and significance of such noise sources have been extensively studied and discussed in many publications.

As the ECG signal data received from the skin are usually contaminated with noise, heart-rate measurement devices equipped with ECG signal analyzing means always include noise filtering means or algorithms in addition to ECG signal processing and analyzing means or algorithms. Digital signal processing techniques are frequently used to perform noise filtering as well as ECG signal processing and analysis because of the many different types of noise as well as the rather complicated ECG signal waveform. However, conventional noise filtering and ECG signal processing techniques are very complicated and require substantial computational overhead which usually means a rather long computational time as well as high energy consumption.

As people are becoming more health conscious, the demands for portable heart rate monitoring or measuring devices and apparatuses have likewise significantly increased. For instance, at the lower cost end of the spectrum, there are numerous wrist-worn type heart-rate monitors which are readily available. These are usually incorporated as part of a wrist-watch and are good examples of one type portable heart-rate monitoring or measuring devices. A typical wrist-worn heart-rate monitoring watch usually includes a wrist strap, a watch casing with a conductive back cover, an ECG sensing electrode mounted on the watch casing and a digital display panel for displaying the time-of-the-day and the heart-rate in beats-per-minute (BPM). Wrist-worn heart-rate monitoring watch have many advantages but because they are usually powered by a single button cell to attain light weight and a compact design, they also often require underlying noise filtering and ECG signal processing algorithms or means so as to not require excessive power consumption to extend battery life.

Wrist-worn heart-rate monitoring watches are well known, for example, in U.S. Pat. Nos. 5,289,824 and 5,738,104. In the wrist-worn heart-rate monitoring watch disclosed in U.S. Pat. No. 5,289,824, the incoming ECG signal data have to pass through numerous filtering stages before subjecting to a QRS complex detection and validation process in order to determine the heart-rate.

U.S. Pat. No. 5,738,104 also discloses a wrist-worn heart-rate monitoring watch including two stages of digital filtering, namely, a first stage of a low pass filter and a second stage of band-pass filter. The digitally filtered ECG signal data are then subject to an enhancement signal processing block which includes a differentiation step followed by a squaring or absolute value operation and are then subject to the calculation of the moving average. A template matching or cross-correlation process on the digitally filtered incoming signal data is then performed to compare or cross check against the results of the enhancement signal processing. The resulting digital data are then analyzed to determine the users' heart-rate. However, the algorithms utilized in most known wrist-worn type heart-rate monitoring watches are often not sufficiently power- and time efficient to satisfy's increasingly stringent consumer demands.

Other types of heart rate monitoring devices which are not wrist-worn are also readily available. These have certain advantages in that they often have greater accuracy and reliability but at the expense of ease of use. Many of these types of devices are better suited to more clinical settings where the user is under the supervision of a health care professional. However, it is not at all practical for a health care professional to constantly monitor a patient for a set period of time, nor for a patient to stay at a clinic (or other locations with health care professionals) for a set period of time, merely for purposes of observing possible symptoms or responses. Instead, ambulatory patients are encouraged to be connected to a monitoring device for a set period of time while going about their regular routine.

Other examples of monitoring devices are Holter recorders which record cardiac signals of an ambulatory patient over a pre-determined period of time. Unlike wrist-worn devices, Holter recorders are typically configured to provide heart activity information, and in particular, electrocardiogram (ECG) recordings over a relatively long period of time. Such recordings permit identification of infrequent and transient disturbances of cardiac rhythm, which may aid in diagnosing patients with vague or intermittent symptoms such as dizziness, blackouts, or fainting spells. Such recordings may also quantify and pinpoint times and/or activities associated with a patient's infrequent symptoms. A physician may be interested not only in the unusual ECG events but also the background rhythm, which may comprise slower or overall responses to influences such as drug treatment, surgery, an implant, or stress. Moreover, a take-home diagnostic device provides more accurate and meaningful ECG recordings since the ambulatory patient is at a home setting (e.g., a natural or real setting) as opposed to an artificial setting (e.g. a doctor's office).

Effectiveness of ECG recording devices involves not only how well cardiovascular signals are measured and recorded, but also its ease of use and cost-effectiveness. Typical Holter recorders, unfortunately, are not inexpensive. Use of diagnostic devices, especially take home diagnostic devices, are also cost-effective and most beneficial for the end-customer (i.e., patients), but may in fact be more costly for medical practitioners due to device purchase and maintenance costs and loss of revenue from future appointments from a given patient. For clinics with budget constraints, spending thousands of dollars for each Holter recorder can be exceedingly expensive. In most cases they are simply are cost prohibitive at the consumer level.

Ease of use of typical Holter recorders is also problematic. The electrode assemblies in typical ambulatory records are reused for many patients, sometimes up to several hundred patients per assembly. The electrode assemblies are not sterilized between uses. Patients even find the idea of having to wear such cables on their skin for up to several days to be unpleasant.

Additionally, typical Holter recorders also tend to be large and thus cumbersome for a patient to carry around at all times during the recording period. And even with the large size, typical Holter recorders can be inefficient in power consumption, which further requires use of large batteries. Finally, due to ease of use issues, it is not uncommon for patients to prematurely end the recording period. Alternatively, patients may be reluctant to even commit to the monitoring because of the degree of discomfort and interference with everyday activities.

The prior art also includes numerous systems wherein ECG data or the like is monitored and/or transmitted from a patient to a particular doctor's office or health service center. Many of these rely on transmission of an audible or sub-audible audio signal which is converted to a electrical signal which is then transmitted to a remote recording station. For example, U.S. Pat. No. 5,735,285 discloses use of a handheld device that converts a patient's ECG signal into a frequency modulated audio signal that may then be analyzed by audio inputting via a telephone system to a selected hand-held computer device or to a designated doctor's office.

Similarly, U.S. Pat. No. 6,264,614 discloses a heart monitor, which is manipulated by the patient to sense a biological function such as a heartbeat, and outputs an audible signal to a computer microphone. The computer processes the audible signal and sends resulting data signals over a network or Internet. U.S. Pat. No. 6,685,633 discloses a heart monitor that a patient can hold against his or her chest. The device outputs an audible signal responsive to the function or condition, such as the beating of the heart, to a microphone connected to a computer. Each of these audio transmissions is limited to transmission of audible sound. In other words, frequency modulated sound transmission at carrier frequencies above that heard by humans, i.e. above 17 kHz, was not contemplated.

U.S. Pat. App. Publication No. 2004/0220487 discloses a system with ECG electrodes which sense ECG electrical signals which are combined and amplitude modulated. The composite signal is transmitted via wire or wirelessly to the sound port in a computing device. A digital band pass filter having a pass band from 19 kHz to 21 kHz is considered; however, there is no consideration of demodulation means at this frequency range using commercially available computing devices. Additionally, the use of sound waves to effect transmission is not contemplated.

U.S. Pat. App. Publication No. 2010/0113950 discloses an electronic device having a heart sensor including several leads for detecting a user's cardiac signals. The leads are coupled to interior surfaces of the electronic device housing to hide the sensor from view. Using the detected signals, the electronic device can then identify or authenticate the user.

U.S. Pat. No. 6,820,057 discloses a system to acquire, record, and transmit ECG data wherein the ECG signals are encoded in a frequency modulated audio tone having a carrier tone in the audio range. However, there is no real consideration of carrier frequencies above about 3 kHz, no consideration of carrier frequencies above the audible, and no consideration of demodulation methods at higher carrier frequencies.

Limitations of the prior art utilizing trans-telephonic and audible acoustic signals include a signal to noise ratio that is diminished by talking or any other noisy activity in the vicinity, thus potentially jeopardizing the integrity of the heart monitoring data signals. Additionally, the audible signals can be heard by anyone in the vicinity of the computer and heart monitor, which can be bothersome to the user as well as to others in the vicinity.

Finally, U.S. Pat. No. 8,301,232 discloses an ECG device which includes an electrode assembly configured to sense heart-related signals upon contact with a user's skin, and to convert the sensed heart-related signals to ECG electrical signals. A converter assembly, integrated with, and electrically connected to the electrode assembly, is configured to receive the ECG electrical signals generated by the sensor and output ECG sound signals through an audio transmitter to a microphone in a computing device within range of the audio transmitter. The converter assembly is further configured to output the ECG signals as an ultrasonic FM sound signal.

Despite some claimed improvements, the transmission of audio signals has inherent limitations and is still subject to acoustical and electronic interference. These and other prior art solutions fail to provide a reliable, inexpensive personal monitoring device that is readily compatible with existing computing devices such as smartphones without transmission of audio signals. It would be advantageous if these issues were addressed in a personal monitoring device transmitting real time physiological data.

Thus, there is a need for a small and lightweight monitoring and diagnostic device for obtaining ambulatory ECG signals. There is also a need for a device that is durable, accurate and relatively inexpensive that can be used in an in-home environment without the need for special electrodes and/or complicated wiring and which provides sufficient data for therapeutic or diagnostic use by health care personnel. There is still a further need for a device that is easy to use, hygienic and portable. Moreover, there is a need for a device that provides simple set-up and data optimization features while still being unobtrusive.

Hence, it is highly desirable if there can be provided an inexpensive, improved signal processing device for heart-rate and ECG determination with both reliable accuracy and low power consumption. Thus, it will be highly desirable if there can be provided simplified schemes or methods for heart-rate measurements suitable for portable, low-power, applications.

### SUMMARY OF THE DISCLOSURE

One aspect of the present invention is a portable apparatus for measuring the heart rate and ECG of a user comprising: a card-like member; a pair of electrodes, a signal processor operatively coupled to the electrodes, memory device for storing and transmitting data within the card-like member and a data processor remote from the card-like member. The user applies his/hers fingers to a circuit board and the voltage generated by the heart muscle is then measured, amplified and sampled by a CPU. In an embodiment data is transferred wirelessly to a remote display device via a wireless protocol where the data is processed and displayed.

Another aspect of the present invention is a portable apparatus for displaying the heart rate and ECG of a user comprising: a card-like member; a pair of electrodes, a signal processor operatively coupled to the electrodes, memory device for storing and transmitting data within the card-like member and a data processor remote from the card-like member and having a display operatively connected to the remote data processor.

Still another aspect of the present invention are a pair of integrated portable devices for measuring and displaying the heart rate and ECG of a user comprising: a credit card-like member; a pair of electrodes, a signal processor operatively coupled to the electrodes, memory device for storing and transmitting data within the card-like member, a data processor remote from the card-like member, a display operably coupled to the processor wherein the data processor further comprises pre-stored calibration data for detecting “out of bounds” data.

In yet another aspect of the present invention, a portable apparatus for measuring a physiological condition is provided comprising: a card-like member; a pair of electrodes, a signal processor operatively coupled to the electrodes, memory device for storing and transmitting data within the card-like member, a data processor remote from the card-like member, a display operably coupled to the processor wherein the data processor transmits the stored and transmitted data to a data recipient. In an embodiment, the data is transferred wireless to a mobile phone or personal computer via NFC, ZigBee, UWB, BlueTooth or other short-range data transmission protocols.

Another aspect of the present invention is a health alert system for monitoring a physiological condition comprising a portable apparatus for measuring a physiological condition comprising: a card-like member; a pair of electrodes, a signal processor operatively coupled to the electrodes, memory device for storing data and transmitting data within the card-like member, a data processor remote from the card-like member, a display operatively connected to the remote data processor and operably coupled to the processor wherein the data processor comprises pre-stored calibration data for detecting “out of bounds” data and wherein the data processor transmits an alert to a data recipient.

Additionally, it will be appreciated that the present disclosure provides a number of advantages over known prior art ECG devices. In terms of manufacturing costs, it is much less expensive as all processing and display is done on a remote display device such as a smart-phone or enabled tablet (collectively “smart phone”), preferably via a wireless connection. This results in a more easily viewed graphic and user interface as compared to a LCD on device. The smaller form factor allows the device of the present invention to be portable and readily carried in a pocket, wallet, handbag or other personal article. Additionally, the wireless connection to a smart-phone enables direct reporting of data to a physician, hospital or data repository without the requirement docking with a computer, although it will be appreciated that in some embodiments the ECG device of the present invention will also be enabled to transmit data both wirelessly and via a wired connection, at the user's option.

Further areas of applicability of the present invention will become apparent from the detailed description provided hereinafter. It should be understood that the detailed description and specific examples, while indicating the preferred embodiment of the invention, are intended for purposes of illustration only and are not intended to limit the scope of the invention.

### BRIEF DESCRIPTION OF THE DRAWINGS

The present invention will become more fully understood from the detailed description and the accompanying drawings, wherein:

FIG. 1 is an overview of a system according to one embodiment of the present invention;

FIG. 2 is a schematic of a device according to an embodiment of the present invention;

FIG. 3 is a schematic of the general operation of the system according to an embodiment of the present invention.

FIG. 4 is top plan view of the device of FIG. 3.

FIG. 5 is a bottom plan view of the device of FIG. 3.

FIG. 6 is a method of using the device of FIG. 3 according to one embodiment of the present invention.

FIG. 7 is a simplified version of the flow chart of FIG. 6.

FIG. 8 is an alternate embodiment of the device of the present invention.

FIG. 9 is a cross sectional representation of an embodiment of the present invention.

FIG. 9a is close up side view of the embodiment shown in FIG. 9.

FIG. 10 is an exemplary final amplification and filtering circuitry of an embodiment of the present invention.

FIG. 11 is a graphic representation of an amplitude and phase response of the final amplification of the filtering circuitry of the present invention.

FIG. 12 is an exemplary feedback loop circuitry of an embodiment of the present invention.

FIG. 13 an exemplary simulation schematic of an embodiment of the present invention.

FIG. 14 is a graphic representation of a feedback simulation of simulation schematic shown in FIG. 13.

FIG. 15 is an alternate implementation of the ECG detection electronics of the present invention.

FIG. 16 is an exemplary power supply and standby mode circuitry of the present invention.

FIG. 17 is a flowchart schematic of the general operation of the system according to an embodiment of the present invention

### DETAILED DESCRIPTION OF THE INVENTION

The following description of the preferred embodiment(s) is merely exemplary in nature and is in no way intended to limit the invention, its application, or uses.

The description of illustrative embodiments according to principles of the present invention is intended to be read in connection with the accompanying drawings, which are to be considered part of the entire written description. In the description of embodiments of the invention disclosed herein, any reference to direction or orientation is merely intended for convenience of description and is not intended in any way to limit the scope of the present invention. Relative terms such as “lower,” “upper,” “horizontal,” “vertical,” “above,” “below,” “up,” “down,” “top” and “bottom” as well as derivative thereof (e.g., “horizontally,” “downwardly,” “upwardly,” etc.) should be construed to refer to the orientation as then described or as shown in the drawing under discussion. These relative terms are for convenience of description only and do not require that the apparatus be constructed or operated in a particular orientation unless explicitly indicated as such. Terms such as “attached,” “affixed,” “connected,” “coupled,” “interconnected,” and similar refer to a relationship wherein structures are secured or attached to one another either directly or indirectly through intervening structures, as well as both movable or rigid attachments or relationships, unless expressly described otherwise. Moreover, the features and benefits of the invention are illustrated by reference to the exemplified embodiments. Accordingly, the invention expressly should not be limited to such exemplary embodiments illustrating some possible non-limiting combination of features that may exist alone or in other combinations of features; the scope of the invention being defined by the claims appended hereto.

The present disclosure is an advanced, compact, handheld credit card-like sized electrocardiograph ECG monitoring device or sensor card that records, stores and/or transmits real time ECG data by means of a wireless data link to an associated data processor having a display for viewing the ECG data. The monitoring device is a personal single lead electrocardiographic monitor for recording and storing real time ECG data for home health use and when transmitted can be processed and viewed by a remote data receiving device and or recipient. The device of the present disclosure is intended for both home based ECG monitoring; when home based ECG monitoring is deemed beneficial by medical professionals or in a clinical setting. The present disclosure is also device intended for self-testing by adult users suitable for healthy people, who wish to develop a self-testing long term routine and for users, who already experience transient symptoms suggesting cardiac conduction abnormality.

The present disclosure also enables voluntary monitoring and also makes it possible for the results to be made available as reference for healthcare professionals, such as general practitioners.

The present disclosure further provides valuable tool for monitoring of the heart during normal daily activities and for compiling a personal cardiac database. Associated software, allows the users to download the recorded data to a variety of data repositories including personal computer via a communications port for the purpose of storing it for personal use or to make it available to their physicians retrospectively. The present disclosure enables both monitoring and self-monitoring as well as recording and is suitable for drug evaluation purposes; cardiac screening and ambulatory home based ECG monitoring. Significantly, the associated display device and software performs the processing of the raw data transmitted by the sensor card. In this regard, data processing overhead is handled primarily, if not exclusively, by the smart-phone or other display device.

The system of the present disclosure comprises an ECG recording device and a related smart-phone mobile application for data processing, analysis, display and reporting. The person applies his/hers fingers to a circuit board and the voltage generated by the heart muscle is then measured, amplified and sampled by a CPU. The data is transferred wireless to a mobile phone via the NFC-protocol, Bluetooth or other short range wireless data transmission protocols. The raw ECG samples are processed and analysed by the mobile software.

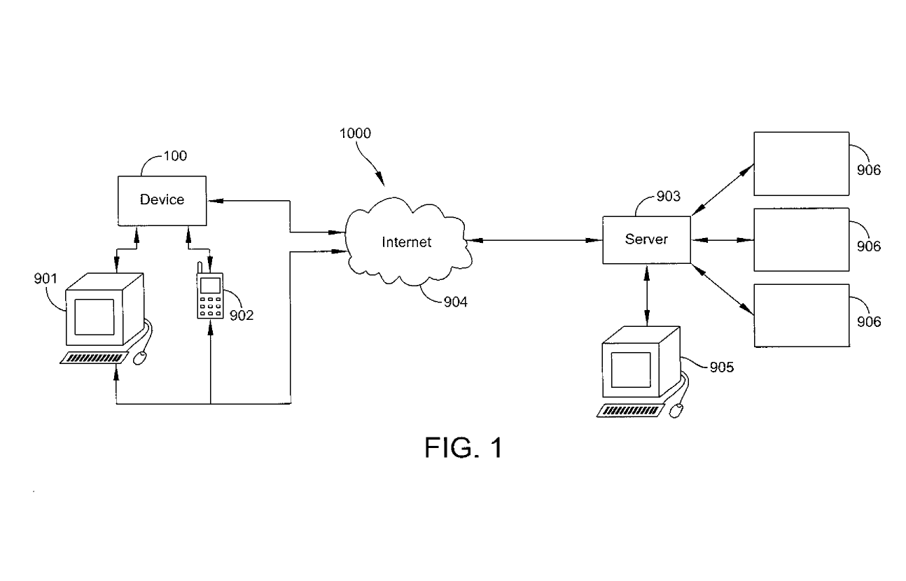

Referring to FIG. 1, a schematic of a system 1000 according to an embodiment of the present invention is illustrated. A device 100 obtains a reading from a user, generates a signal relating to measured physiological parameter, in this case an ECG measurement, calculates values relating to the received reading and transmits the values and corresponding information to an external device. In an embodiment, the external device can be a personal computer 901, a mobile communication device 902, a remote server 903 and/or remote personal computer 905. The transmission of the measured values can be by means of a wireless communication device located within the device or a wired connection to the external device. In the embodiment shown, the device transmits filtered but raw data to an associated display device where the raw data is processed, analyzed and displayed.

Pursuant to the disclosure, the processing, analysis and display of the data occurs remote from device 100. This arrangement minimizes, if not largely eliminates, data processing overhead on device 100 and shifts it to an external device with associated software and/or display capabilities. In an alternate embodiment, device 100 may simply store the actual measured values and corresponding information until the device 100 is sent to a centralized data processing and diagnostics site where the device 100 is scanned and the information retrieved. In an alternate embodiment, the physiological parameter may be wirelessly transmitted to a personal computer 901 or mobile communication device 902 at the location of the user, and then transmitted through the internet 904 to a remote server 903 for viewing on a personal computer 905, such as a personal computer located at a physician or doctor's office. Optionally, raw data may be stored in one or more databases 906 for later retrieval, analysis or for historical reference purposes.

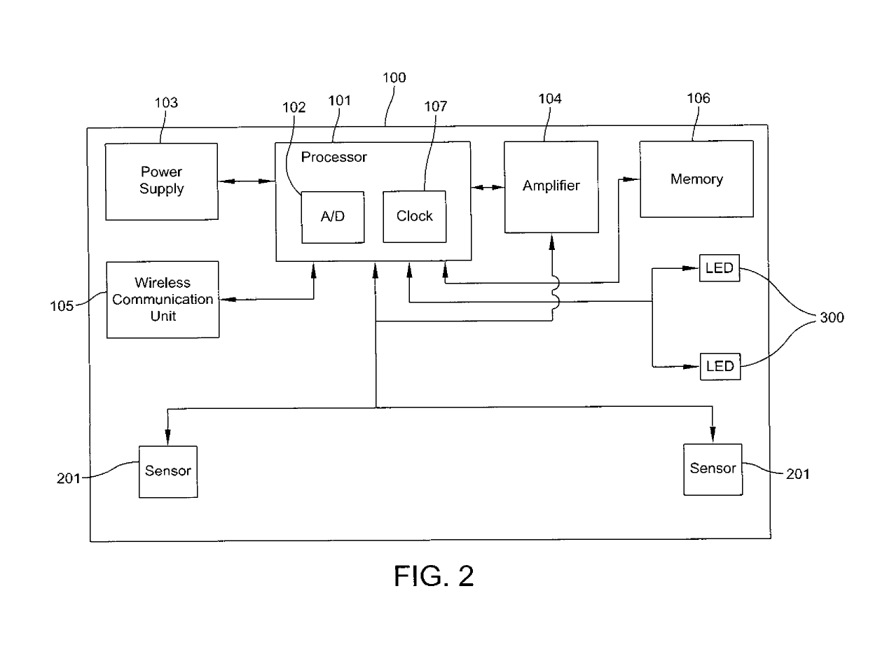

Referring to FIG. 2, a schematic of the device 100 according to an embodiment of the present invention is illustrated. The device comprises a processor 101, a power supply 103, filter/amplifier unit 104, a wireless communication unit 105, memory unit 106, electrodes 200 and at least one indication device 300. A suitable filter for use in the present disclosure is typically a basic analog low pass anti-aliasing filter to reduce A/D sampling artifacts. More advanced filtering can be done by the remote signal processor to keep device complexity and cost down.

In the exemplified embodiment, the processor 101 comprises signal-conditional means, data processing means, data acquisition means, and analog-to-digital converter (A/D) 102, and an internal clock 107. The processor 101 is operably coupled to and configured to control the interaction of the power supply 103, filter/amplifier unit 104, the wireless communication unit 105, memory 106, electrodes 201 and the at least one indication device 300. Specifically, processor 101 must be configured to the ECG reading specifics of electrodes 201 configured within the device 100. The clock 107 is configured to provide time-keeping means to allow each measurement of the device 100 to be time-stamped and stored in the memory unit 106. The power supply 103 is operably coupled to and configured to supply power to the processor 101, amplifier unit 104, the wireless communication unit 105, memory 106, electrodes 201 and at least one indication device 300. The memory unit 106 is operably coupled to the processor 101 and configured to store data and to transfer data to wireless communications unit 105 via processor 101. In one embodiment, the memory unit 106 may be a non-volatile memory unit. In other embodiments, a second internal clock or timer inside processor 101 can be used to convert the A/D data at a fixed rate.

Wireless communication unit 105 is operably coupled to and configured to transmit data wirelessly to an external device. In one embodiment, the wireless communication device 105 comprises an integrated planar antenna. Further, in one embodiment, the wireless communication device 105 uses radio frequency identification (RFID) to communication with the external device. The wireless communication device 105 may use active, passive, or semi-passive RFID technologies. In alternate embodiments, the wireless communication device 105 may be a Bluetooth® enabled device or ZigBee® enabled device. Further, in other alternate embodiments disclosed herein, the wireless communication device 105 may be a device that uses other wireless protocol for wireless communication not limited to Bluetooth or ZigBee protocols.

It should be noted that in some alternate embodiments, the wireless communication device may be temporarily inactivated and the device may also comprise various ports for wired connections to the external device. Also, since the information being transmitted by the wireless communication device 105 may be confidential, optional cryptographic operations can be performed prior to data exchange, so that only a legitimate receiver can decrypt and verify the data retrieved from the device 100. It will be appreciated that in other alternative embodiments, data may be transmitted via wire or other non-wireless means as well as by wireless transmission between device 100 and personal computer 901 or mobile communication device 902 at the location of the user, and then transmitted through the internet 904 to a remote server 903 for viewing on a personal computer 905, such as a personal computer located at the patient's residence, a medical facility or doctor's office.

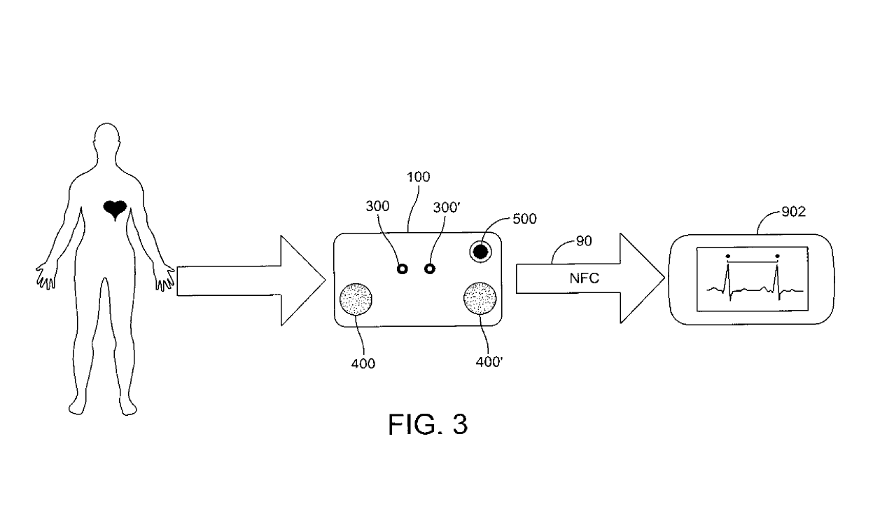

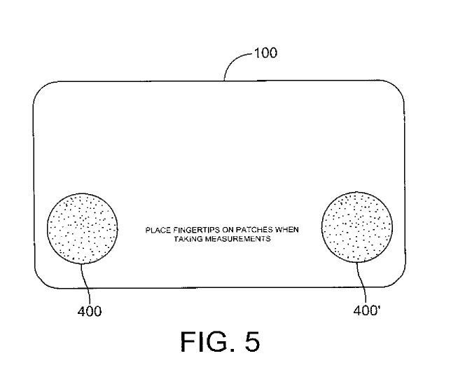

FIG. 3 is a schematic of the general operation of the system according to an embodiment of the present invention. The user will interact with device 100 by holding it between opposing thumb and forefingers of the right and left hand in a light pinch grasp. Device 100 is shown having a pair of electrodes 400 positioned on the face surface of the card which will typically receive the user's thumb pads, on the lower surface of device 100 are a complimentary pair of electrodes that will typically receive the fingertips of the user as will be shown more clearly in FIG. 5. It will be understood that in many of the embodiments disclosed, other fingers (as well as other body parts including the legs) can be used as long as the conventional two lead is maintained.

In an embodiment, electrodes 400 (and 400′) may be positioned within shallow recesses to allow a user to more easily position his or her thumbs and fingers in the electrode with minimal visual cues. At least one indicator 300 provides a visual signal of the operational status of the device. In one embodiment indicator 300 is a pair of LED light of different colors, for example, green 300 and red 300′, that will indicate whether an acceptable measurement was made by the user. In this embodiment, indicator 300 will light if the measurement is good and indicator 300′ will light if the measurement is not acceptable or otherwise needs to be remade. It will be appreciated that a single multicolored LED may be employed as indicator 300 and may include other signaling means such as sound generating devices and/or vibrational or haptic technology devices, for a tactile feedback which takes advantage of the user's sense of touch by applying forces, vibrations, or motions to the user. In other embodiments, device 100 may also include optional power switch 500 which the user may manually activate prior to taking an ECG measurement. After a successful measurement and processing of the raw ECG information data packet 90 is transmitted via NFC to display device 902 where it may be read, analyzed and/or further transmitted to secondary or tertiary remote locations.

Additionally, in yet other embodiments, electrodes 400 and 400′ may be supplemented by other electrodes which may be temporarily affixed to a user's body by known adhesive means. These supplemental electrodes may be enabled to wirelessly transmit sampled data to device 100. It will be appreciated that in these embodiments, device 100 would be adapted to accept input both from electrodes 400 and 400′ and wirelessly from a plurality of electrodes and that number and placement of electrodes can be increased for even greater accuracy and could effectively function as 3, 5, 6 or even 12 lead ECG monitors. In such embodiments, the preferred combination of wireless electrodes would be paired with or coupled to device 100 to form the desired multiple lead configurations. Moreover, in these and other embodiments, it will be further appreciated that ECG monitoring could be selectively operational to be more or less constant in a manner similar to Holter monitoring and/or triggered by user activation of device 100 via a user interface, such as a power button/on-off switch, when activated by gripping the device for a preset time period or as at timed intervals.

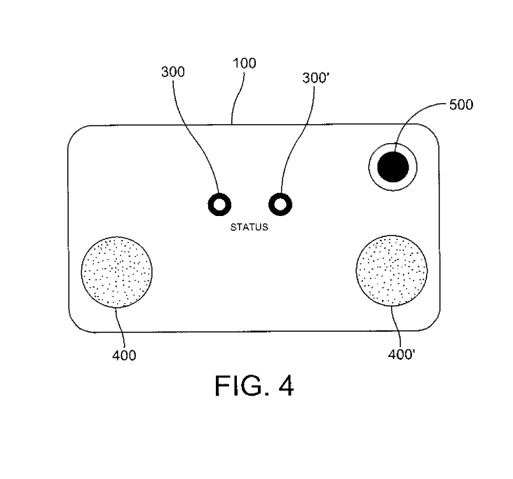

FIG. 4. is a top plan view of device 100 of FIG. 3 shown having a pair of electrodes 400 positioned on the face surface of the card which will typically receive the user's thumb pads, on the lower surface of device 100 are a complimentary pair of electrodes that will typically receive the fingertips of the user as will be shown more clearly in FIG. 5. In the embodiment depicted, electrodes 400 (and 400′) may be positioned within shallow recesses to allow a user to more easily position his or her thumbs and fingers in the electrode with minimal visual cues. A pair of LED light of different colors, for example, green 300 and red 300′, that will indicate whether an acceptable measurement was made by the user. In this embodiment, indicator 300 will light if the measurement is good and indicator 300′ will light if the measurement is not acceptable or otherwise needs to be remade. Device 100 also includes power switch 500 which the user activates prior to taking an ECG measurement. Device 100 may also include brief instructions on the face of the card to direct the user in the operation of the device.

FIG. 5. is a bottom plan view of device 100 of FIG. 4 shown having a pair of electrodes 400′ positioned on the lower surface of the card which will typically receive the user's fingertip pads. In the embodiment depicted, electrodes 400′ are positioned within shallow recesses to allow a user to more easily position his or her thumbs and fingers in the electrode with minimal visual cues. Device 100 may also include brief instructions on the lower of the card to direct the user in the operation of the device.

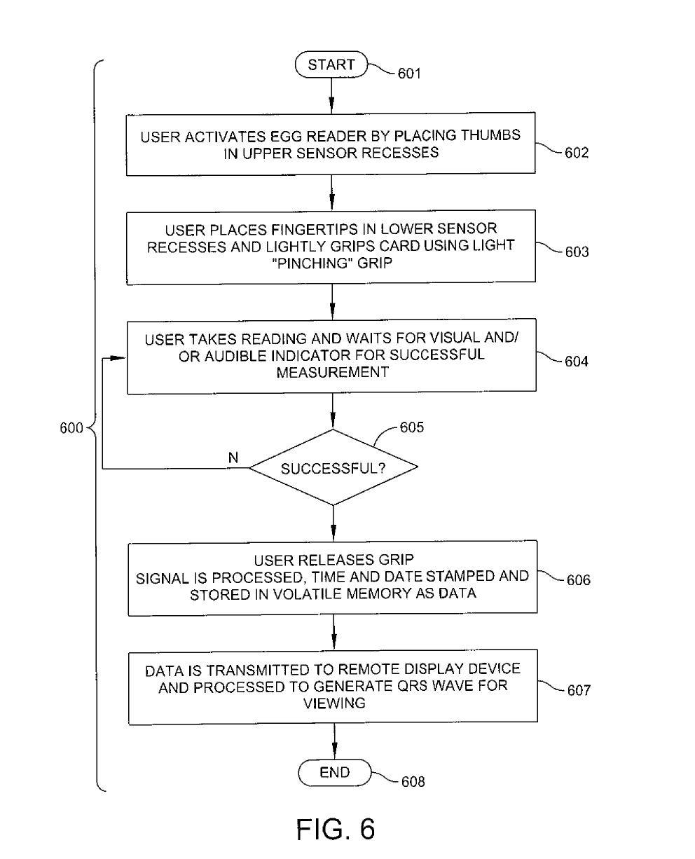

FIG. 6. is a flow chart showing the general operation of the ECG monitoring device of the present invention. Device 100 begins at start 601 by activating the device via a start switch or by grasping the card-like member and holding it via electrodes to energize a dormant circuit at 602. At 603 the user places fingertips in lower electrode recesses and lightly grips card using a “pinching” grip. In step 604 the user takes reading and waits for the predetermined time period for visual and/or audible indicator for successful measurement. Typically, a pre-set time period for initial reading from 10 seconds to 60 seconds will be required in order to commence an accurate measurement. Step 605 tests whether the measurement was successful. If not user will return the user to step 604 and retake the measurement. If the measurement was successful, the user will release its grip; and at step 606 the signal is then time and date stamped and stored in volatile memory as raw data. At step 607 the raw data is transmitted to one or more remote display device and processed to generate the ECG signal waveform. Typically, an ECG waveform with many QRS complexes will be displayed and it is the QRS that is the common subject of inquiry; however, it will be appreciated that the QRS is just a part of the complete waveform. At step 608 the process is completed and the device returns to dormant or powered down state.

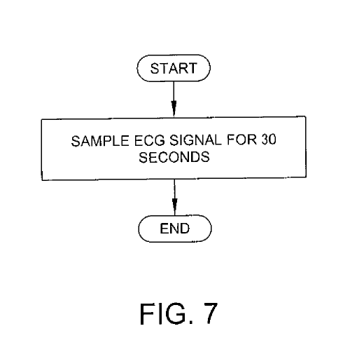

FIG. 7 is a simplified version of the flow chart of FIG. 6. In its most elemental operational mode device 100 begins at START 601 by activating the device via a start switch; in step 604, the ECG signal is sampled for 30 seconds; the signal is then time and date stamped and stored in volatile memory as raw data, transmitted to one or more display devices and at step 608 the process is completed and the device returns a powered down state at END.

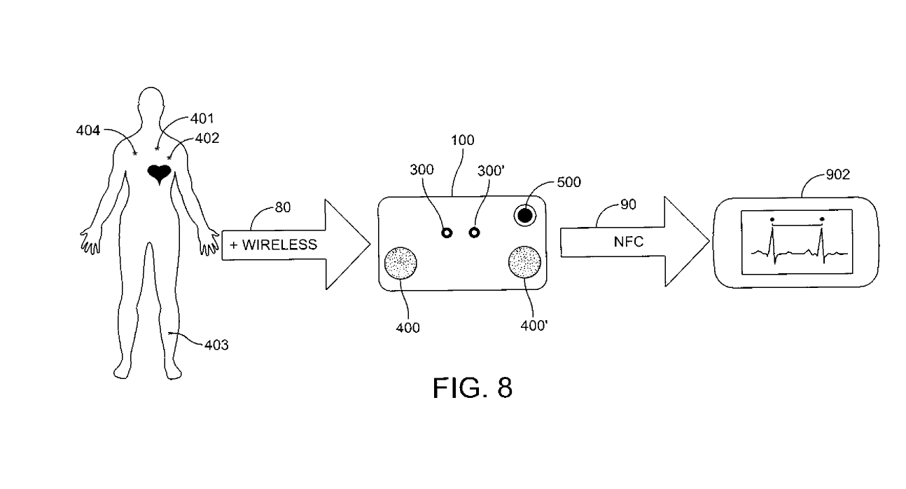

FIG. 8 is an alternate form of the present invention in which electrodes 400 and 400′ may be supplemented by wireless electrodes 401, 402, 403, 404, etc. These wireless electrodes may be temporarily affixed to a user's body by known adhesive means. In this embodiment, the electrodes are enabled to wirelessly transmit sampled data to device 100′ using dedicated wireless protocols 80 such as RFID, NFC, Bluetooth, ZigBee, UWM and other low power and/or short range protocols. The wireless electrodes are preferably powered by low power battery circuits to reduce power consumption.

It will be also appreciated that in these embodiments, device 100′ is a transceiver adapted to accept input both from electrodes 400 and 400′ and to wirelessly receive transmitted data from a plurality of paired wireless electrodes and then transmit all of the sampled data via wireless protocol 90 to display device 902 for processing. As used herein, a transceiver comprises both a transmitter and a receiver which is combined and share common circuitry or a single housing. It will be understood that the number and placement of the wireless electrodes can be increased for even greater accuracy which would allow device 100′ to effectively function as 3, 5, 6 or even 12 lead ECG monitors when used in conjunction with electrodes 400 and 400′. In such embodiments, the preferred combination of wireless electrodes are paired with or coupled to device 100′ to form the desired multiple lead configurations without significantly changing the form factor of the card-like member.

The receiving and transmission of the measured data values is by means of the wireless communication unit located within the device and/or by means of a wired connection to the external device. In the embodiment shown, device 100′ transmits filtered but largely unprocessed or raw data to an associated display device where the raw data is processed, analyzed and displayed by an external device thereby lessening processing overhead on device 100′. The external device can include a personal computer 901, a mobile communication device 902, a remote server 903 and/or remote personal computer 905.

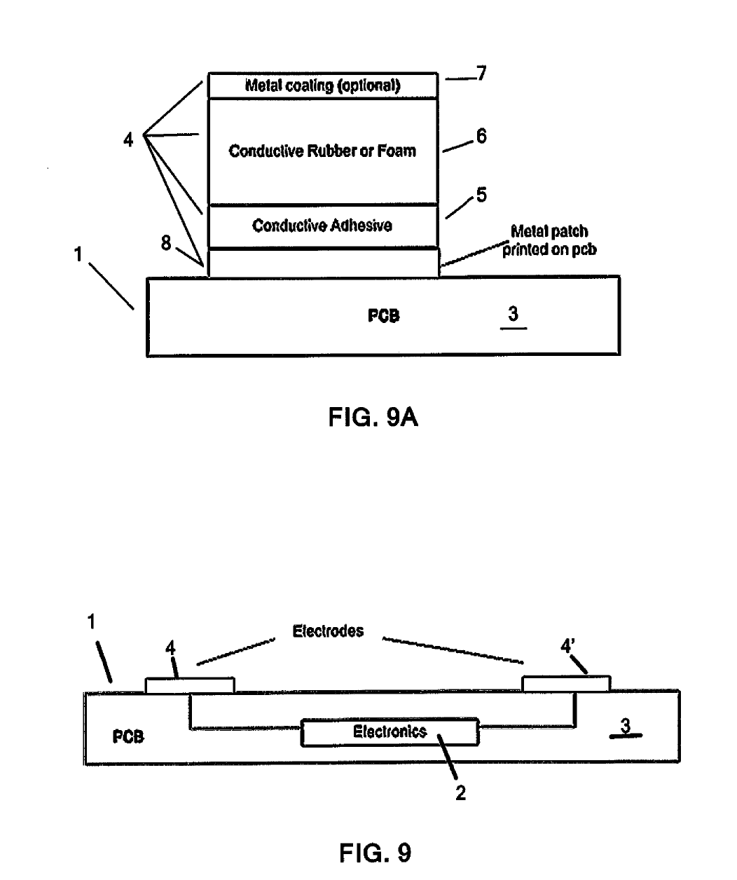

A close-up and a magnified cross section of an embodiment the ECG device 1 of the present invention are shown with the key component parts is shown in FIGS. 9 and 9a. It will be understood that the electronics 2 are embedded on or integrally formed on a printed circuit board 3 including all of the key electronic components all of which will typically be encased in a plastic housing having approximately same peripheral dimensions of a conventional credit card or larger depending on the internal electronics.

As used herein, “card-like member” shall be understood to include any generally planar, approximately credit-card to index card sized housing in which the sensors and associated electronics 2 may be encased. Electrodes 4 and 4′ may be positioned in such a manner to accessible to the opposite thumbs of a user and may be raised, flush or positioned slightly lower than the upper and/or lower surfaces of the card-like member. In the embodiment shown, electrodes 4 and 4′ may be fabricated with layers of electro-conductive materials including but not limited to conductive adhesives 5, conductive rubber or foam 6 with an optional metal coating 7. For lowest cost, the electrode sensors may optionally be implemented as etched conductive metal (e.g. Au/Ni, Au, Ag, Cu) plates 8 directly on the PCB. In order to reduce the electrical contact impedance to the skin, an electrolytic solution (e.g. NaCl or Na+ Cl− (aq) in H2O or H+ CO3− in H2O) can be dropped on the plates prior to applying fingers.

Alternative electrode designs use planar patches made of flexible conductive material to improve skin contact. The flexibility of the rubber increase contact area as because of the adaption to the skin structure. The flexibility also stabilizes the impedance during movements or tremors of the users fingers as the contact area between finger and electrode is more constant compared to the rigid metal plates. The flexible patches can be realized by conductive rubber or foam only. Alternatively, a layer of conductive rubber or foam coated with a very thin layer (micro-meters) of metal can be used. The connection to the PCB is made via conductive adhesives and etched metal plates.

In one embodiment the system of the present disclosure comprises an ECG recording device in generally flat, planar credit card sized format and separate application, typically via a smart phone mobile for data processing, analysis, display and reporting. In an embodiment, the data is retrieved from the card via a Near Field Communications (NFC), Bluetooth and/or other wireless protocol.

The sensor card is a thumb ECG signal recorders and make use of standard ECG measurement principles. That is, the electrical changes which are caused by heart muscle activity are measured via the skin. Instead of using electrodes connected to chest and extremities, a user connects to the electrode by placing the thumbs on two electrode patch areas integrated on the card. Referring to standard ECG terminology, a single lead measurement setup is achieved.

The ECG signal is measured via integrated electrodes and then amplified to suitable range (typically ×100). Basic analog low-pass anti-aliasing filter is applied and signal is digitized using an A/D Converter, sampled and stored to a non-volatile memory. Typically, the signal is sampled at 250 Hz for 30 secs. The memory is read out from the device via the wireless interface. In other words, the sensor device is a basic ECG electrodes sampling device with wireless data reporting means.

The ECG signal samples are read from the sensor device. Digital signal processing is applied to remove mains hum (50/60 Hz), base line wandering etc. The resulting signal is used for display of the ECG waveform and further processing, validation and analysis such as QRS Detection for calculation of R-R intervals, ST interval, Heart Rate. The processing device processes the data, stores and displays the results.

In one embodiment of the present disclosure, to start a measurement, a user first activates a power-on button and holds the card between the opposing fingers and thumbs on the user's right and left hands. One such method would be to place each thumb pad on the two electrode patch areas integrated on the upper surface of the card while simultaneously placing the one pair (i.e. right and left hand) of the remaining fingers on each of the corresponding patches on the lower surface of the card to form a “pinching grip” to hold the card and activate power to the internal circuitry.

For ease of reference, each electrode pair may have upper and lower surfaces designated, for example as P1 and P2 for one electrode and P3 and P4 for the other electrode. This arrangement ensures that there is a greater likelihood of contact sufficient to sample and measure ECG voltages. A green LED embedded on the card surface indicates sampling is in progress. A red LED embedded on the card will light if a reading error occurred. Optionally, the visual LED indicators can be accompanied by an audible signalling device to alert the user of the operational status of the ECG card during the reading phase of operation. The ECG voltage signal is sampled for 30 seconds and stored to the cards memory. When the measurement is completed, the sample data is transferred via wireless transmission such as Bluetooth or NFC to a smart-phone having software for processing and displaying the ECG data.

It will be understood that other embodiments may optionally include an on/off switch to provide power to the circuitry prior to starting measurement of the ECG. Alternate ways to activate the device can also be employed. It may be possible for the user to activate the device through a push-button or properly from gripping the electrodes for a sustained period of time. At the time of activation the red LED will light up. The user will have a time frame, typically 2-4 seconds to place his/her fingers on the sensor pads before the data acquisition of the ECG data will commence. During the data acquisition the green LED will light up indicating that sampling is in progress.

In an embodiment, when a measurement is taken, digital filtering of data is necessary to remove 50/60 Hz hum and other artifacts. An instrumentation amplifier is used to receive the small amplitude voltage signals generated by the heart muscle. The amplifier provides a built-in amplification of from 5 to 10 times and has a large degree of common mode suppression. The human body acts as an antenna inserting a large amount of 50/60 Hz noise into the circuit together with the weak ECG signal. This noise is mostly common-mode noise which is suppressed by the instrumentation amplifier. The data is then validated and preliminarily analysed to confirm that the measured signals are sufficiently clean to make further analysis. If the data read is un-usable, the user will be sent either a visual or audible signal to warn the user that second reading is necessary.

It will be appreciated that the data does not necessarily have to be analysed locally. The data qualification by the device may simply be a range check to make sure that the collected data was mostly within the measurement range of the ADC. The measured data is then transmitted for remote processing.

Even with the mode suppression present in the instrumentation amplifier there may still be a large 50/60 Hz noise contribution in the raw data. An easy way to suppress this is to set the ADC data conversion frequency to a multiple of the power line frequency. It is then possible by means of a simple running average to greatly reduce the 50/60 Hz noise that passes through the instrumentation amplifier. For example, by using a 300 Hz data acquisition frequency, which is a multiple of both 50 Hz and 60 Hz, by averaging 6 data points when a 50 Hz line frequency is present and 5 data points when a 60 Hz line frequency is present, the power line interference is filtered out. This type of filtering may be done on the device or later after transfer to the mobile device or PC.

In use, a patient/user applies his/hers fingers to the sensor device and the voltage generated by the heart muscle is then measured, amplified and sampled by a CPU. The data is transferred wirelessly to a mobile phone, for example, via the NFC-protocol, Bluetooth or other similar wireless protocol. It will be appreciated that the person being measured is holding electrode 4, including upper and lower contact surfaces P1 and P2 with one hand and electrode 4′, including upper and lower contact surfaces P3 and P4 with the other. The signal is then fed into the amplifier.

One focus of the application is to present data to be able to detect arrhythmias or other cardiac anomalies. The analysis and calculation data generally proceeds using the following parameters. In processing the data, an Average Heart Rate (AHR) is first determined (as measured by a 30 sec period)—then, QRS detection and subsequent R-R interval timings (instantaneous heart rates) are calculated.

In an embodiment, resistors may be needed to allow for a common mode path. In the absence of the resistors the inputs would float and the output values would saturate.

### Final Amplification and Filtering

In an embodiment, the amplitude of the signal fed to the CPU ADC is preferably as close to the middle of the dynamic range of the ADC as possible to make the best use of the full dynamic range of the ADC. It therefore needs to be further amplified after the instrumentation amplifier and this is done using the circuit shown by example in FIG. 10.

Generally, the amplifier is a standard inverting amplifier with 43 dB gain (147 times) with a first degree low pass filter. The −3 dB cutoff frequency of the filter is ˜34 Hz and helps to avoid unwanted signals being measured.

The cut-off frequency is set by the C₈ and R₁₆ components and is found from the equation:

$${f}_{c} = \frac{1}{2 π \cdot {R}_{16} \cdot {C}_{8}} .$$

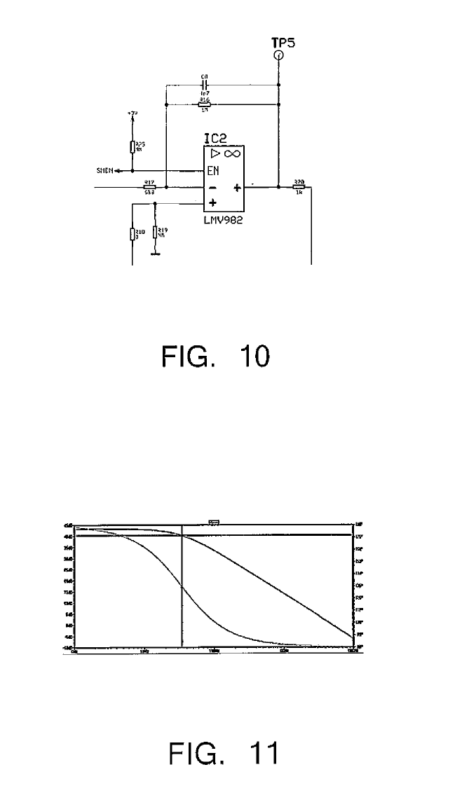

An amplitude and phase response of the final amplification stage using the DC-offset elimination feedback is generally shown in FIG. 11.

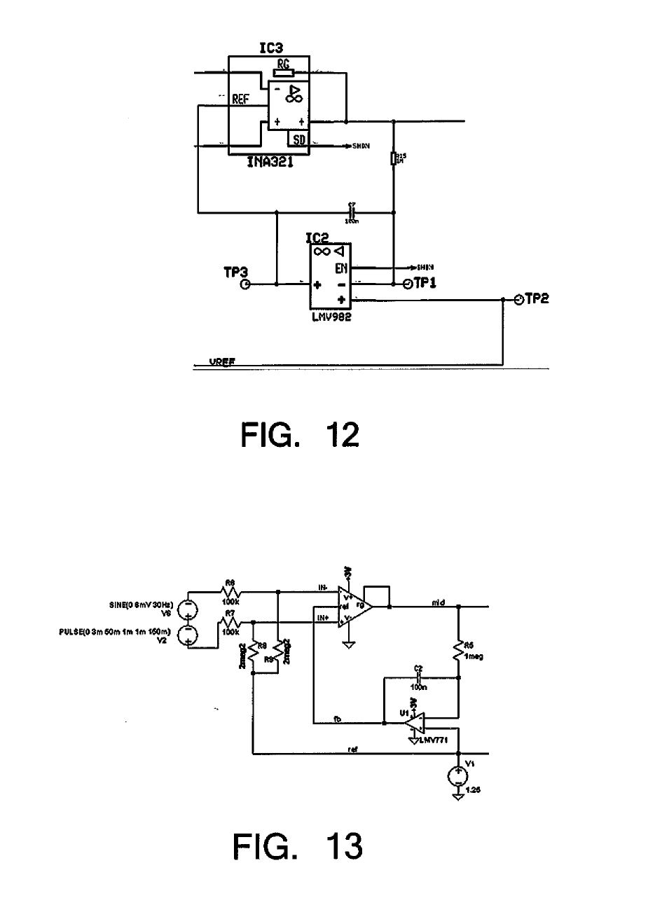

It will be further appreciated that the differences in contact resistance between the right and left hand of the person being measured results in a DC-voltage which appears on the output of the instrumentation amplifier. With the large amplification of the IC2:2 the amplifier output can easily saturate resulting in a distorted signal. The feedback loop shown by example, in FIG. 12 is therefore used to adjust the REF-input of IC3 so that the DC-offset on the IC3 output comes back to V_{REF} level.

The output of the IC3 is therefore compared to the V_{REF} voltage using an inverting integrator IC2:1. The principle is best demonstrated the simulation.

A useful signal is simulated with a sine wave 6 mV, 30 Hz which is fed into the instrumentation amplifier. This signal appears on the output amplified 5 times centered around 1.25V (V(mid)—mid graph in FIG. 14 showing feedback simulation). A DC-offset of 3 mV is then applied using the V2 after 50 ms. The output then jumps up this offset amplified 5 times. The output of the inverting integrator is then shown in the bottom graph. It started off at ˜1.25 V but then as the pulse comes it slowly drops trying to adjust the output back to be centered on 1.25V.

The opposite thing happens at 200 ms in the simulation when the DC-offset is removed. The output (V(mid)) first jumps down but is adjusted back after a while as the feedback output is increased.

The output preferably shall be maximum 1.25V (range of ADC) as generally described above. The offset is therefore set to approximately 0.5V.

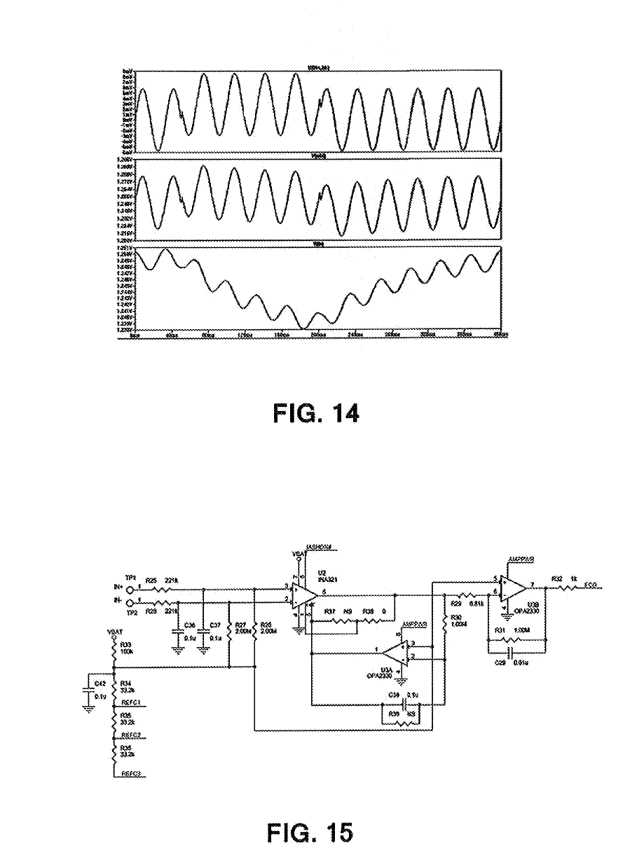

FIG. 15 shows an alternate implementation of the ECG detection electronics. TP1 and TP2 are the electrodes that a user touches. The combination of R28/C36 and R25/C37 provide a low pass filter with a 3 dB point of 7 Hz. This allows signals from the human heart beat to pass while providing a first filter stage to remove the higher frequency signals due to power line pickup. Some of the higher frequency components of the heartbeat, such as the QRS complex, may be slightly attenuated, but this device main purpose is to measure statistics of the beat to beat interval, which occurs at a much lower frequency of 7 Hz. It will be appreciated that it is also possible to change these values if desired.

### Bias Supply

Referring to FIG. 15, it will be appreciated that REFC1, REFC2 and REFC3 are 3 different locations which can be grounded by a microcontroller. This allows the setting of a variety of bias signals to the fingers, but most typically REFC3 is grounded, while REFC1 and REFC2 are left floating. This results in a reference potential halfway the battery voltage, so that the dynamic range of the instrumentation amplifier is used most efficiently.

### Power Control

The microcontroller also provides the signals IASHDN# and AMPPWR, which will power up the instrumentation amplifier, U2, or the operational amplifiers, U3, respectively. By controlling the power through the microcontroller it can be ensured that the lowest amount of power is used by the device while it is not active.

### Instrumentation Amplifier

There are many possible instrumentation amplifiers. In one alternate embodiment the INA321, U2, from Texas Instruments, which has a Common Mode Rejection Ratio (CMRR) of 94 dB is used. The datasheet contains a reference design for ECG measurements from which the implementation in FIG. 15 is derived.

### Feedback Loop

Small differences in contact resistance between the right and left hand of the person being measured results in a DC-voltage which appears on the output of the instrumentation amplifier. With the large amplification of U2, the amplifier output can easily saturate resulting in a distorted signal. U3A implements a feedback loop into the reference of the instrumentation amplifier to remove this effect. A similar implementation is shown in the INA321 data sheet.

### Final Gain Stage

U3B is the final gain stage amplifier. Its gain is determined by the ratio of R31 and R29 which in this particular example is 147. Signals with frequencies over 16 Hz are attenuated.

### Preferred Power Supply and Standby Mode

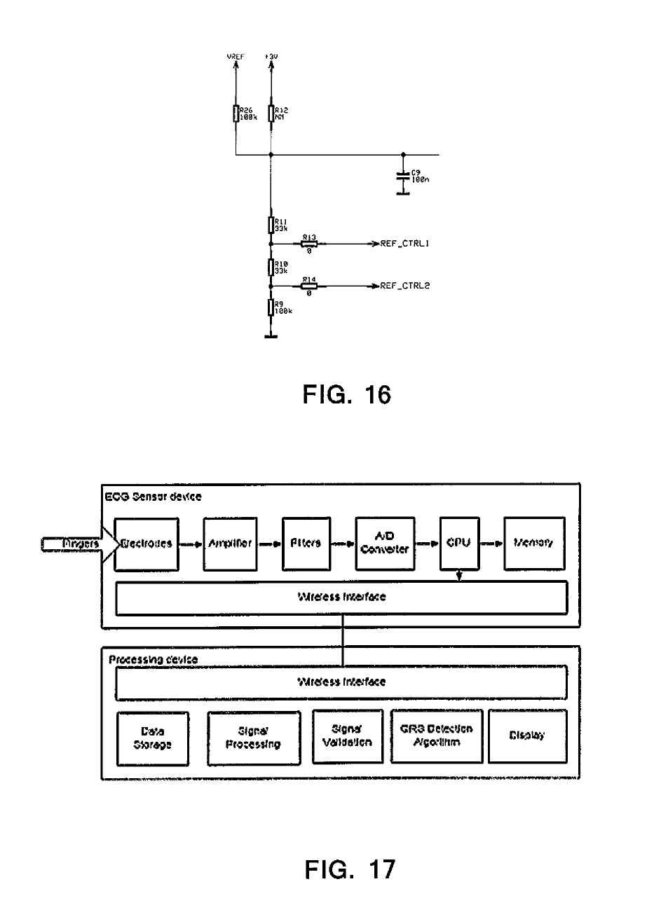

In an embodiment shown in FIG. 16, the power to the circuit comes from a 3V primary Lithium battery. The voltage from the battery directly feeds the ASIC. When the no measuring is done, the ASIC turns of the voltage to the OP-amps which are fed directly from an IO-pin (IO5). In this way, the standby current consumption is made very low.

### Reference Voltage Generation

The reference voltage is used to create a virtual ground for the op-amp chain to center the waveform around. The reference voltage can be changed between three different values by two digital outputs (on the ASIC). It originates from the VREFBUF output of the ASIC (named VREF in below) which is holding 2.4 V constantly over battery life.

As used throughout, ranges are used as shorthand for describing typical values that are within the range. Any value within the range can be selected as the terminus of the range. In addition, all references cited herein are hereby incorporated by referenced in their entireties. In the event of a conflict in a definition in the present disclosure and that of a cited reference, the present disclosure controls.

In use the ECG device of the present disclosure generally operates according the schematic flowchart representation as shown in FIG. 17.

The following embodiments are exemplary. Although the specification may refer to “an”, “one” or “some” embodiment(s) in several locations, this does not necessarily mean that each such reference is to the same embodiment(s), or that the feature only applies to a single embodiment. Single features of different embodiments may also be combined to provide other embodiments.

Several embodiments of the present invention are specifically illustrated and described herein. However, it will be appreciated that modifications and variations of the present invention are covered by the above teachings and within the purview of the appended claims without departing from the spirit and intended scope of the invention.

What is claimed is:

1. A portable apparatus for measuring the electrocardiogram of a user comprising:
a generally planar card-like member;
a microprocessor within the card-like member;
a power source;
at least one pair of electrodes for capturing data indicative of an electrical signal of a heart, wherein the at least one pair of electrodes are fixed to the card-like member and operably coupled to the microprocessor; and
a wireless transmitter for sending data to a remote processing device.

2. The apparatus of claim 1 wherein the apparatus further comprising a memory device is operably coupled to the microprocessor, the microprocessor storing the data within the memory device.

3. The apparatus of claim 2 further comprising an indicator configured to indicate whether the data captured by the microprocessor indicates whether the electrode registered an accurate measurement and/or whether the sensor registered an inaccurate measurement.

4. The apparatus of claim 1 further comprising an indicator fixed to the card-like member and operably coupled to the processor to provide an indication to the user of a status state selected from the group comprising: whether the sensor is in the off or on mode; whether the sensor has sufficient memory to store multiple readings or whether an associated wirelessly coupled display device is in proximity.

5. The apparatus of claim 1 wherein the at least one pair of sensors are located within a depression on an upper surface of the card-like member.

6. The apparatus of claim 1 wherein the at least one pair of sensors are located within a depression on an upper surface of the card-like member and further wherein a second pair of electrodes are located on the lower surface of the card-like member substantially beneath said at least one pair of electrodes.

7. A portable apparatus for measuring the electrocardiogram of a user comprising:
a generally planar card-like member;
a microprocessor housed within the card-like member,
a power source operative connected to said microprocessor
at least one pair of electrodes for capturing data indicative of an electrical signal of a heart, wherein the at least one pair of electrodes are fixed to the card-like member and operably coupled to the microprocessor;
at least one electrode for capturing data indicative of an electrical signal of a heart, wirelessly connected to the card-like member and operably coupled to the microprocessor
a wireless transceiver for wirelessly receiving and transmitting data to a remote processing device.
